(19) United States
(12) Patent Application Publication
(10) Pub. No.: US 2022/0087890 A1
(43) Pub. Date: Mar. 24, 2022

(54) Vision Impaired Navigating Assembly

(71) Applicant: Claudian Rachel, Brooklyn, NY (US)

(72) Inventor: Claudian Rachel, Brooklyn, NY (US)

(21) Appl. No.: 17/029,811

(22) Filed: Sep. 23, 2020

(51) Int. Cl. A61H 3/06; H04R 1/02; G01S 19/42; G01S 19/14; G01C 21/20; A61B 5/1172; A45B 3/08

(52) U.S. Cl. CPC A61H 3/061; H04R 1/028; G01S 19/42; G01S 19/14; H04W 4/80; A61H 3/068; A61B 5/1172; A45B 3/08; G01C 21/20

(57) ABSTRACT

A vision impaired navigating assembly for guiding a visually impaired user while walking includes a cane that is gripped by a visually impaired user to assist the visually impaired user with walking. A biometric scanner is coupled to the cane to scan the visually impaired user's fingerprint. A first transceiver is integrated into the cane and the first transceiver is in wireless communication with a global positioning system (gps) for gps navigation. A second transceiver is integrated into the cane and the second transceiver is in wireless communication with a personal electronic device. A speaker is coupled to the cane to emit audible sounds outwardly therefrom related to gps navigation for guiding the visually impaired user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to navigating devices and more particularly pertains to a new navigating device for guiding a visually impaired user while walking.

### (2) Description of Related Art Including Information Disclosed Under 37 CFR 1.97 and 1.98

The prior art relates to navigating devices including a communication device that incorporates wireless communication into a variety of everyday devices. The prior art discloses a walking stick that includes gps location capabilities and that is in wireless communication with surveying equipment for performing a geological survey. The prior art discloses a variety of walking canes that have gps navigation capabilities. In at least one instance the walking canes are able to be synchronized with a personal electronic device.

### BRIEF SUMMARY OF THE INVENTION

An embodiment of the disclosure meets the needs presented above by generally comprising a cane that is gripped by a visually impaired user to assist the visually impaired user with walking. A biometric scanner is coupled to the cane to scan the visually impaired user's fingerprint. A first transceiver is integrated into the cane and the first transceiver is in wireless communication with a global positioning system (gps) for gps navigation. A second transceiver is integrated into the cane and the second transceiver is in wireless communication with a personal electronic device. A speaker is coupled to the cane to emit audible sounds outwardly therefrom related to gps navigation for guiding the visually impaired user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a vision impaired navigating assembly according to an embodiment of the disclosure.

FIG. 2 is a top view of an embodiment of the disclosure.

FIG. 3 is a front view of an embodiment of the disclosure.

FIG. 4 is a right side phantom view of an embodiment of the disclosure.

FIG. 5 is a perspective view of an alternative embodiment of the disclosure.

FIG. 6 is a schematic view of an embodiment of the disclosure.

### DETAILED DESCRIPTION OF THE INVENTION

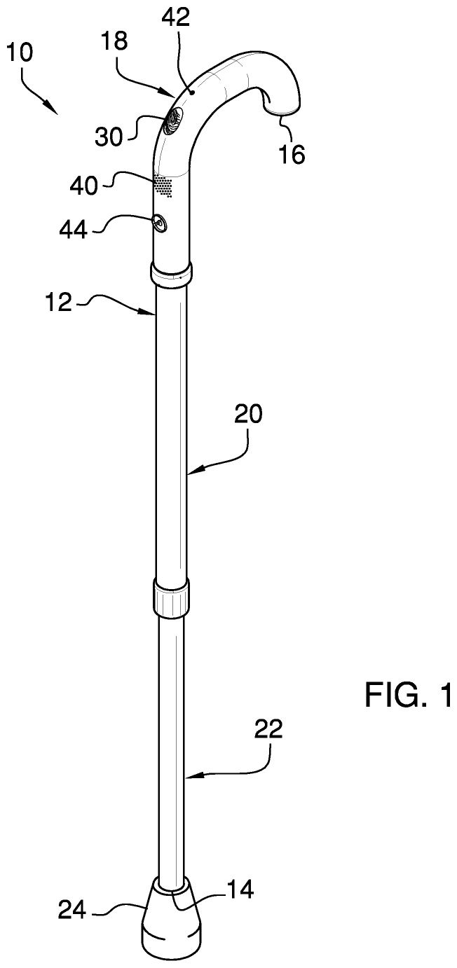

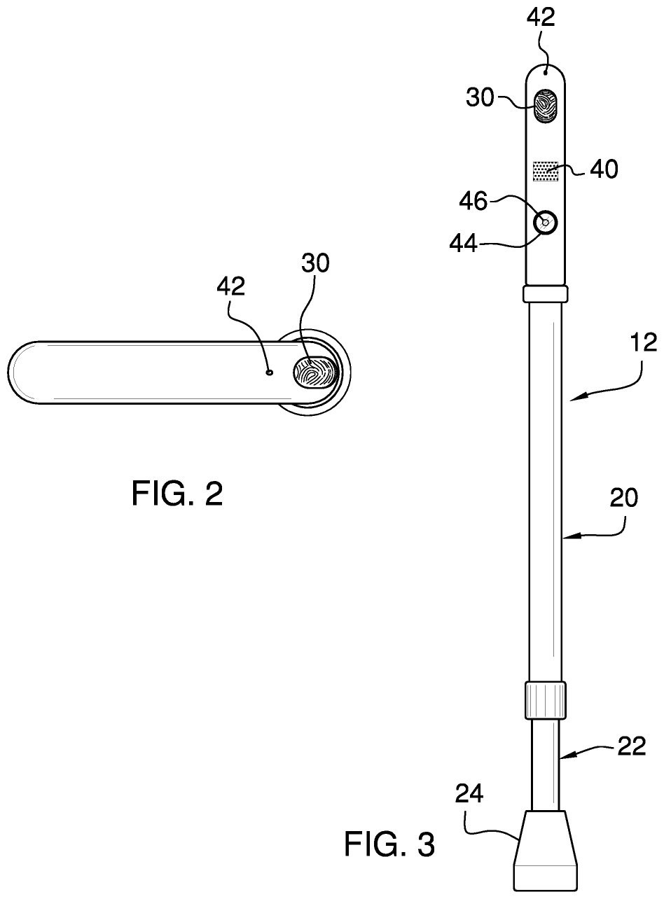

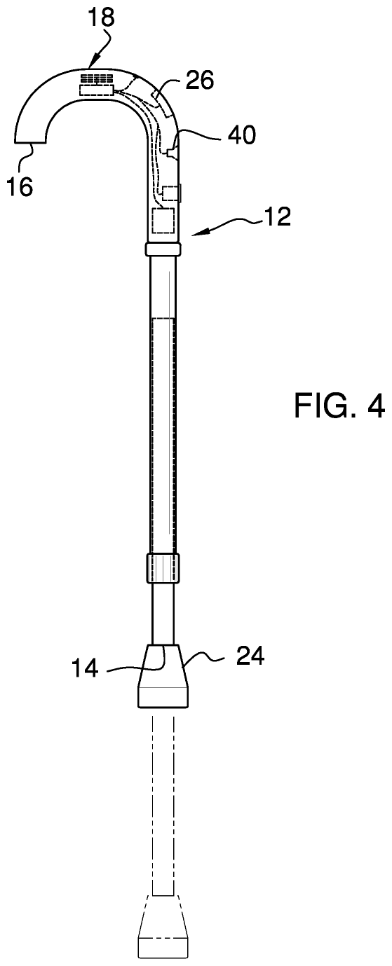

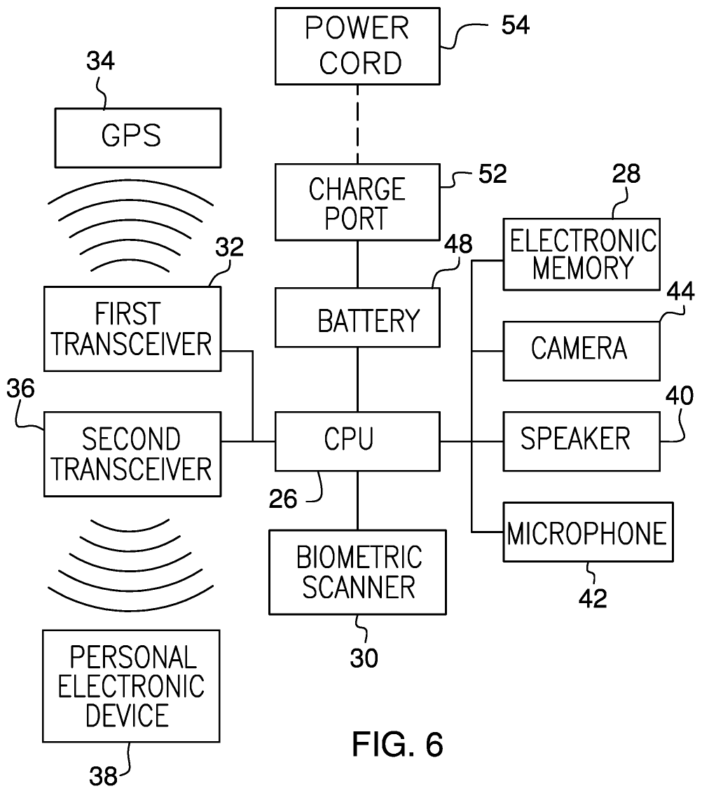

With reference now to the drawings, and in particular to FIGS. 1 through 6 thereof, a new navigating device embodying the principles and concepts of an embodiment of the disclosure and generally designated by the reference numeral 10 will be described.

As best illustrated in FIGS. 1 through 6, the vision impaired navigating assembly 10 generally comprises a cane 12 that is gripped by a visually impaired user to assist the visually impaired user with walking. The cane 12 has a first end 14 and a second end 16, and the cane 12 has a bend 18 positioned adjacent to the second end 16. Thus, the second end 16 is directed toward the first end 14 such that the cane 12 has a J shape. The cane 12 may comprise a first portion 20 that slidably receives a second portion 22 such that the cane 12 has a telescopically adjustable length. A foot 24 may be coupled to the first end 14 of the cane 12 for enhancing engaging a support surface upon which the visually impaired user is walking.

A control circuit 26 is integrated into the cane 12 and the control circuit 26 receives an actuate input. An electronic memory 28 is integrated into the cane 12 and the electronic memory 28 is electrically coupled to the control circuit 26. The electronic memory 28 stores data comprising the visually impaired user's fingerprint. Additionally, the electronic memory 28 stores data comprising operational software and the electronic memory 28 stores data comprising a plurality of pre-determined verbal commands. The electronic memory 28 may comprise digital memory such as RAM or other similar type of miniaturized data storage.

A biometric scanner 30 is coupled to the cane 12 and the biometric scanner 30 scans the visually impaired user's fingerprint. The biometric scanner 30 is electrically coupled to the control circuit 26. The control circuit 26 receives the actuate input when the biometric scanner 30 scans a fingerprint that matches the visually impaired user's fingerprint stored in the electronic memory 28. Moreover, the biometric scanner 30 is disposed on the bend 18 to enhance the visually impaired user's ability to locate the biometric scanner 30. The biometric scanner 30 may be an electronic fingerprint scanner of any conventional design.

A first transceiver 32 is integrated into the cane 12 and the first transceiver 32 is electrically coupled to the control circuit 26. The first transceiver 32 is in wireless communication with a global positioning system (gps) 34 to receive the physical location of the cane 12 from the gps 34. The first transceiver 32 is turned on when the control circuit 26 receives the actuate input. Additionally, the first transceiver 32 may comprise a radio frequency transceiver or the like.

A second transceiver 36 is integrated into the cane 12 and the second transceiver 36 is electrically coupled to the control circuit 26. The second transceiver 36 is in wireless communication with a personal electronic device 38, such as a smart phone or other similar device with wireless communication capabilities. In this way an assistant to the visually impaired user can be informed of the visually impaired user's location. The second transceiver 36 is turned on when the control circuit 26 receives the actuate input. Additionally, the second transceiver 36 may comprise a radio frequency transceiver or the like and the second transceiver 36 may employ Bluetooth communication protocols.

A speaker 40 is coupled to the cane 12 to emit audible sounds outwardly therefrom and the speaker 40 is electrically coupled to the control circuit 26. The speaker 40 emits audible directions related to gps navigation to guide the visually impaired user while the visually impaired user is walking. In this way the visually impaired user can navigate city streets or other geographic location without assistance. Additionally, the speaker 40 is turned on when the control circuit 26 receives the actuate input.

A microphone 42 is coupled to the cane 12 to detect audible sounds and the microphone 42 is electrically coupled to the control circuit 26. The microphone 42 communicates the pre-determined commands to the electronic memory 28 when the visually impaired user speaks the pre-determined verbal commands. In this way the microphone 42 facilitates voice control of the operational software in the electronic memory 28. The microphone 42 is turned on when the control circuit 26 receives the actuate input and the microphone 42 may be an electronic microphone 42 of any conventional design.

A camera 44 is coupled to the cane 12 for capturing imagery. The camera 44 is electrically coupled to the control circuit 26 and the camera 44 is turned on when the control circuit 26 receives the actuate input. The camera 44 may comprise a digital camera of any conventional design, and the camera 44 includes a lens 46 that is mounted to the cane 12. The camera 44 may facilitate the assistant to the visually disabled user to view imagery of the area in which the visually disabled user is walking.

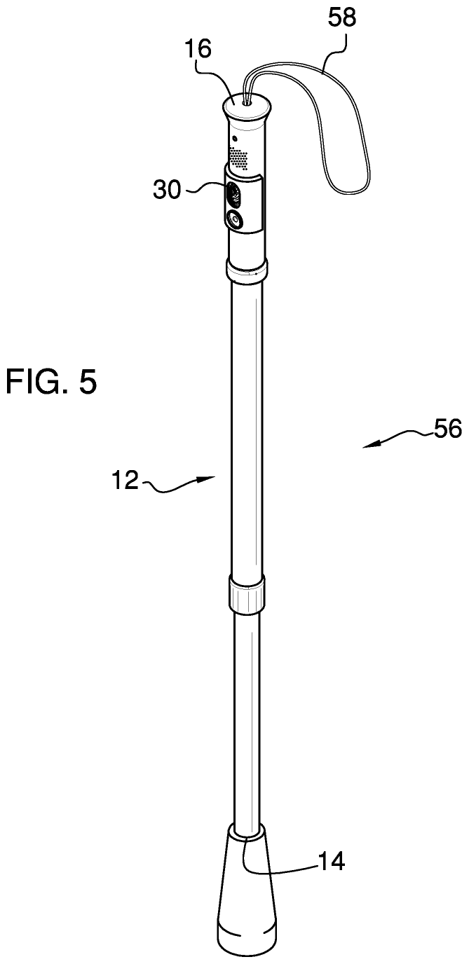

A power supply 48 is integrated into the cane 12 and the power supply 48 is electrically coupled to the control circuit 26. The power supply 48 comprises a rechargeable battery 50 that is positioned inside the cane 12 and the rechargeable battery 50 is electrically coupled to the control circuit 26. The power supply 48 comprises a charge port 52 that is integrated into the cane 12 to receive a power cord 54 from a charger. Additionally, the charge port 52 is electrically coupled to the rechargeable battery 50 for charging the rechargeable battery 50. In an alternative embodiment 56 as is shown in FIG. 5, the cane 12 may be elongated between the first end 14 and the second end 16. A wrist strap 58 may be coupled to the second end 16 of the cane 12 for securing the cane 12 to the visually impaired user.

In use, the visually impaired user touches the biometric scanner 30 to unlock the control circuit 26 in the cane 12. In this way the microphone 42 is actuated to record verbal commands spoken by the visually impaired user, such as gathering gps 34 navigation data for a selected destination. The speaker 40 emits verbal directions for the visually impaired user to follow thereby facilitating the visually impaired user to navigate city streets without assistance. Additionally, the camera 44 facilitates the assistant to view imagery captured so the assistant can help the visually impaired user to identify landmarks or buildings.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A vision impaired navigating assembly for facilitating gps directions for a visually impaired user, said assembly comprising:
a cane being gripped by a visually impaired user wherein said cane is configured to assist the visually impaired user with walking;
a biometric scanner being coupled to said cane wherein said biometric scanner is configured to scan the visually impaired user's fingerprint;
a first transceiver being integrated into said cane, said first transceiver being in wireless communication with a global positioning system (gps) wherein said first transceiver is configured to receive the physical location of said cane from the gps;
a second transceiver being integrated into said cane, said second transceiver being in wireless communication with a personal electronic device; and
a speaker being coupled to said cane wherein said speaker is configured to emit audible sounds outwardly therefrom, said speaker emitting audible directions related to gps navigation wherein said speaker is configured to guide the visually impaired user while the visually impaired user is walking.

2. The assembly according to claim 1, wherein:
said cane has a first end and a second end, said cane having a bend being positioned adjacent to said second end having said second end being directed toward said first end such that said cane has a J shape; and
said biometric scanner is disposed on said bend wherein said biometric scanner is configured to enhance the visually impaired user's ability to locate said biometric scanner.

3. The assembly according to claim 1, further comprising a control circuit being integrated into said cane, said control circuit receiving an actuate input.

4. The assembly according to claim 3, further comprising an electronic memory being integrated into said cane, said electronic memory being electrically coupled to said control circuit, said electronic memory storing data comprising the visually impaired user's fingerprint, said electronic memory storing data comprising operational software, said electronic memory storing data comprising a plurality of pre-determined verbal commands.

5. The assembly according to claim 3, wherein said biometric scanner is electrically coupled to said control circuit, said control circuit receiving said actuate input when said biometric scanner scans a finger print that matches the visually impaired user's fingerprint stored in said electronic memory.

6. The assembly according to claim 3, wherein said first transceiver is electrically coupled to said control circuit, said first transceiver being turned on when said control circuit receives said actuate input.

7. The assembly according to claim 3, wherein said second transceiver is electrically coupled to said control circuit, said second transceiver being turned on when said control circuit receives said actuate input.

8. The assembly according to claim 3, wherein said speaker is electrically coupled to said control circuit, said speaker being turned on when said control circuit receives said actuate input.

9. The assembly according to claim 4, further comprising a microphone being coupled to said cane wherein said microphone is configured to detect audible sounds, said microphone being electrically coupled to said control circuit, said microphone communicating the pre-determined commands to said electronic memory when the visually impaired user speaks the pre-determined verbal commands wherein said microphone is configured to facilitate voice control of the operational software in said electronic memory, said microphone being turned on when said control circuit receives said actuate input.

10. The assembly according to claim 3, further comprising a camera being coupled to said cane wherein said camera is configured to capture imagery, said camera being electrically coupled to said control circuit, said camera being turned on when said control circuit receives said actuate input.

11. The assembly according to claim 3, further comprising a power supply being integrated into said cane, said power supply being electrically coupled to said control circuit, said power supply comprising:
a rechargeable battery being positioned inside said cane, said rechargeable battery being electrically coupled to said control circuit; and
a charge port being integrated into said cane wherein said charge port is configured to receive a power cord from a charger, said charge port being electrically coupled to said rechargeable battery for charging said rechargeable battery.

12. A vision impaired navigating assembly for facilitating gps directions for a visually impaired user, said assembly comprising:
a cane being gripped by a visually impaired user wherein said cane is configured to assist the visually impaired user with walking, said cane having a first end and a second end, said cane having a bend being positioned adjacent to said second end having said second end being directed toward said first end such that said cane has a J shape;
a control circuit being integrated into said cane, said control circuit receiving an actuate input;
an electronic memory being integrated into said cane, said electronic memory being electrically coupled to said control circuit, said electronic memory storing data comprising the visually impaired user's fingerprint, said electronic memory storing data comprising operational software, said electronic memory storing data comprising a plurality of pre-determined verbal commands;
a biometric scanner being coupled to said cane wherein said biometric scanner is configured to scan the visually impaired user's fingerprint, said biometric scanner being electrically coupled to said control circuit, said control circuit receiving said actuate input when said biometric scanner scans a finger print that matches the visually impaired user's fingerprint stored in said electronic memory, said biometric scanner being disposed on said bend wherein said biometric scanner is configured to enhance the visually impaired user's ability to locate said biometric scanner;
a first transceiver being integrated into said cane, said first transceiver being electrically coupled to said control circuit, said first transceiver being in wireless communication with a global positioning system (gps) wherein said first transceiver is configured to receive the physical location of said cane from the gps, said first transceiver being turned on when said control circuit receives said actuate input;
a second transceiver being integrated into said cane, said second transceiver being electrically coupled to said control circuit, said second transceiver being in wireless communication with a personal electronic device, said second transceiver being turned on when said control circuit receives said actuate input;
a speaker being coupled to said cane wherein said speaker is configured to emit audible sounds outwardly therefrom, said speaker being electrically coupled to said control circuit, said speaker emitting audible directions related to gps navigation wherein said speaker is configured to guide the visually impaired user while the visually impaired user is walking, said speaker being turned on when said control circuit receives said actuate input;
a microphone being coupled to said cane wherein said microphone is configured to detect audible sounds, said microphone being electrically coupled to said control circuit, said microphone communicating the pre-determined commands to said electronic memory when the visually impaired user speaks the pre-determined verbal commands wherein said microphone is configured to facilitate voice control of the operational software in said electronic memory, said microphone being turned on when said control circuit receives said actuate input;
a camera being coupled to said cane wherein said camera is configured to capture imagery, said camera being electrically coupled to said control circuit, said camera being turned on when said control circuit receives said actuate input; and
a power supply being integrated into said cane, said power supply being electrically coupled to said control circuit, said power supply comprising:
a rechargeable battery being positioned inside said cane, said rechargeable battery being electrically coupled to said control circuit; and
a charge port being integrated into said cane wherein said charge port is configured to receive a power cord from a charger, said charge port being electrically coupled to said rechargeable battery for charging said rechargeable battery.
